(19) United States
(12) Patent Application Publication
(10) Pub. No.: US 2018/0021499 A1
(43) Pub. Date: Jan. 25, 2018

(54) UREA SEQUESTRATION COMPOSITIONS AND METHODS

(71) Applicant: Cabot Corporation, Boston, MA (US)

(72) Inventors: Michael J. Natan, Weston, MA (US); Agathagelos Kyrlidis, Cambridge, MA (US)

(21) Appl. No.: 15/547,963

(22) PCT Filed: Feb. 1, 2016
(86) PCT No.: PCT/US2016/015935

§ 371 Date: Aug. 1, 2017

Related U.S. Application Data
(60) Provisional application No. 62/113,098, filed on Feb. 6, 2015.
(60) Provisional application No. 62/113,106, filed on Feb. 6, 2015.

(51) Int. Cl. A61M 1/16; A61M 1/36; B01D 15/08; C07C 275/02; B01J 20/28; C01B 32/182; C01B 32/23; C02F 1/28; A61M 1/34; B01J 20/20

(52) U.S. Cl. CPC A61M 1/1696; A61M 1/3486; A61M 1/3679; B01D 15/08; B01J 20/20; B01J 20/205; B01J 20/28057; C01B 32/182; C01B 32/23; C02F 1/286; C07C 275/02; B01D 2253/102; B01D 2253/306; B01D 2257/40; B01D 53/04

(57) ABSTRACT

Graphene-based materials for sequestering urea from aqueous solutions are provided. The graphene-based materials include graphene aggregates as well as graphene oxides.

### CROSS REFERENCE TO RELATED APPLICATIONS

This application is a §371 national phase application of PCT/US2016/015935, filed on Feb. 1, 2016, which claims priority to U.S. Provisional Application No. 62/113,098, filed on Feb. 6, 2015, and U.S. Provisional Application No. 62/113,106, filed on Feb. 6, 2015. The above-referenced applications are incorporated by reference in their entirety.

### FIELD OF THE INVENTION

The present disclosure relates to methods and materials for removing urea from aqueous solutions, and, in particular, to graphene-based materials useful for removing urea from biological fluids.

### BACKGROUND

Urea is a small, highly polar molecule that, by virtue of its polarity and capability to participate in hydrogen bond formation, is highly soluble in water (>400 mg/ml) and in protic organic solvents such as methanol, ethanol, and glycerol. While the role of urea in biochemistry is essential, and it is an important molecule industrially, including as a source of nitrogen for fertilizer and as a polymer precursor, it is often important for urea to be removed from fluid solutions.

### SUMMARY

In one aspect, a method is provided, the method comprising contacting a fluid comprising urea with a mass of graphene-based material particles, sorbing at least a portion of the urea into or onto the graphene-based material particles to produce a graphene-based material/urea complex and reducing the level of urea in the fluid wherein the amount of urea in the graphene-based material/urea complex is greater than 25 mg urea per gram of graphene-based material. The fluid can be selected from at least one of an aqueous fluid, water, whole blood, blood plasma, processed blood, preserved blood, serum, plasma, clotted blood, anti-clotted blood, centrifuged blood, hematocrit, biological filtrate, ultrafiltrate, dialysate, extracellular fluids, intracellular fluids, interstitial fluids, lymphatic fluids, transcellular fluids, urine and urine-derived fluids. The amount of urea associated with the graphene-based material/urea complex can be greater than 50, greater than 100, greater than 250, greater than 500, or greater than 700 mg urea per gram of graphene-based material. In some cases, the concentration of urea in the fluid is reduced by greater than 10, 25, 50 75, 90, 99, 99.9, 99.99, 99.999, 99.9999, or 99.99999 percent by weight. The method may include agitating, stirring, shaking, sonicating, flowing, cooling and/or heating a suspension of the graphene-based material particles in the fluid. The method may include flowing the fluid through a bed comprising graphene-based material particles. The graphene-based material can be a graphene oxide having an atomic ratio of carbon to oxygen of from 20:1 to 1.5:1, 10:1 to 1.5:1, 5:1 to 1.5:1, 3:1 to 1.5:1, 2:1 to 1.5:1, 10:1 to 2:1 or 5:1 to 2:1. The graphene-based material can be a graphene aggregate. The method can include removing at least one non-urea component of the fluid with graphene-based materials, activated carbon and/or modified activated carbon.

In another aspect, a method is provided, the method comprising contacting a dialysate with graphene-based material, the dialysate comprising urea, sorbing at least a portion of the urea on or in the graphene-based material to form a graphene-based material/urea complex, reducing the concentration of urea in the dialysate by greater than 25%, wherein the graphene-based material/urea complex comprises at least 10% urea by weight. The method can also include contacting the dialysate with activated carbon or with graphene-based material and the method of contacting may be selected from dispersing graphene-based material particles in the dialysate, passing the dialysate through a bed comprising graphene-based material particles, passing the dialysate through a membrane comprising graphene-based material and passing the dialysate through a column comprising graphene-based material. The graphene-based material can be graphene aggregates or graphene oxide and at least a portion of the graphene-based material/urea complex can be formed through intercalation. The method can further comprise removing urea from the graphene-based material/urea complex and may be used to purify a fluid derived from one or more tissues of a patient exhibiting kidney dysfunction. The tissue can be blood and the sorbing can occur between 0° and 50° C., between 23° and 37° C. and/or at a pH of between 4 and 8. The method can include sterilizing the graphene-based material.

In another aspect, a method is provided, the comprising contacting a fluid comprising urea with a mass of an intercalation host having interlayer spacing of between 2 and 15 Å, sorbing at least a portion of the urea into or onto the intercalation host to produce an intercalated complex, and reducing the level of urea in the fluid wherein the amount of urea in the intercalated complex is greater than 25, 50, 100, 500 or 700 mg urea per gram of intercalation host.

In another aspect, a method is provided, the method comprising contacting a fluid comprising urea with a mass of an intercalation host having interlayer spacing equivalent to the size of a urea molecule, +/−10%, 20%, 30% or 40%, sorbing at least a portion of the urea into or onto the intercalation host to produce an intercalated complex, and reducing the level of urea in the fluid wherein the amount of urea in the intercalated complex is greater than 25, 50, 100, 500 or 700 mg urea per gram of intercalation host. The intercalation host can have an interlayer spacing of between 2 and 6 Å, and may be selected from graphene, graphene oxide, graphite oxide or mixtures thereof. The intercalation host can have a nitrogen BET surface area of greater than 2600 m²/g, greater than 1300 m²/g, greater than 850 m²/g, greater than 650 m²/g, greater than 530 m²/g or greater than 440 m²/g, and a total pore volume of pores greater than 1 nm in size of less than 0.01 cm³/g, less than 0.1 cm³/g, less than 0.5 cm³/g, less than 1.0 cm³/g or less than 2.0 cm³/g when measured using mercury porosimetry or nitrogen desorption. The intercalation host can have an interlayer spacing of 2 to 15 Å, 4 to 12 Å, 7 to 11 Å, 8 to 11 Å, 8 to 10 Å, 6 to 9 Å, 5 to 8 Å, 4 to 8 Å, 2 to 8 Å, 2 to 6 Å, 3 to 6 Å, 8 to 12 Å, 9 to 12 Å or 10 to 14 Å and may be one or more planar layers comprising or consisting essentially of sp² hybridized carbon atoms.

Where applicable to the methods herein, the temperature of the fluid during sorption can be in the range of 0° C. to 50° C., 10° C. to 40° C., 20° C. to 40° C., 30° C. to 40° C., less than 40° C., less than 30° C., less than 20° C., less than 10° C., greater than 0° C., greater than 10° C., greater than 20° C. or greater than 30° C. The pH of the fluid during sorption is in the range of 3 to 10, 4 to 10, 5 to 10, 5 to 9, 6 to 9, 6 to 8, 7 to 8, less than 9, less than 8, less than 7, less than 6, greater than 3, greater than 5, greater than 7 or greater than 8. The fluid can comprise at least one of, or a mixture of, whole blood, blood plasma, processed blood, preserved blood, serum, plasma, clotted blood, anti-clotted blood, centrifuged blood hematocrit, dialysate, dialysis-derived fluids, hemodialysate, peritoneal dialysate, plasmapheresis-derived fluids, diafiltration-derived fluids, ultrafiltration-derived fluids, filtration-derived fluids, fluids generated by diffusion-based processes, fluids generated by convection-based processes, fluids generated by processes under laminar flow, fluids generated by processes under turbulent flow, or any combination thereof. The graphene-based materials (GM) or intercalation host can sorb urea and physically exclude larger materials while allowing the passage of water. The fluid can be returned to a patient, and the method may include treating the blood of a patient in need of dialysis. The fluid being treated can be associated with a patient showing symptoms of kidney disease or kidney failure, and the method can reduce the concentration of urea in the blood of a patient exhibiting signs of kidney disease or kidney failure. The fluid may comprise at least one of, or a mixture of, whole blood, blood plasma, processed blood, preserved blood, serum, plasma, clotted blood, anti-clotted blood, centrifuged blood and hematocrit. In some cases, the fluid comprises dialysate.

In another aspect a composition is provided, the composition comprising graphene-based material particles and urea sorbed to the graphene-based material wherein the ratio of urea to graphene-based material is greater than 1:10 by weight. The graphene-based material can include graphene aggregates or graphene-based material oxide and greater than 90% of nitrogen content in the composition may be in the form of urea. The composition may also comprise activated carbon or modified activated carbon. The urea can comprise hydrogen bonded urea aggregates, the urea in the form of dimers, trimers or n-mers where n is from 4 to 50.

In another aspect, a device comprising graphene-based material is provided, the device configured to accept a fluid comprising urea. The device can be a dialysis cartridge. The cartridge can also include activated carbon and may include a graphene-based material/urea complex. The graphene-based material can include graphene aggregates and/or graphene oxide. The dialysis cartridge can include a filter capable of filtering high molecular weight components from a fluid, and the filter can comprise graphene-based material.

In another aspect, a graphene-based material/urea complex comprising at least 10% urea by weight is provided. The graphene-based material/urea complex can be used to store urea by sorbing and/or desorbing urea to or from the complex.

In another aspect, a method is provided, the method comprising exposing a graphene-based material sorbent to an atmosphere comprising urea, sorbing urea into or onto the GM sorbent, and reducing the concentration of urea in the atmosphere.

In another aspect, a method is provided, the method comprising contacting a multi-layered graphene-based material with urea, intercalating the urea between adjoining layers in the graphene-based material, and exfoliating the graphene-based material. The exfoliating can occur in the absence of any exfoliating agents other than urea. The graphene-based material can be contacted with the urea in an aqueous system.

### BRIEF DESCRIPTION OF THE DRAWINGS

In the figures:

FIG. 1 provides the chemical structure and associated functional groups of an embodiment of a graphene oxide sheet;

FIG. 2 illustrates the hydrogen bonding that can occur between graphene oxide and water;

FIG. 3 is a graph showing thermogravimetric and differential scanning calorimetry analysis of a graphite oxide example;

FIG. 4 is a photocopy of a TEM image of an embodiment of pH 3 exfoliated graphene oxide;

FIG. 5 is a photocopy of a TEM image of an embodiment of pH 9 exfoliated graphene oxide;

FIG. 6 provides proton NMR experimental results illustrating an absence of any urea breakdown products;

FIG. 7 provides carbon NMR experimental results illustrating an absence of any urea breakdown products;

FIG. 8 provides UV-vis results for two different experimental urea solutions, illustrating that urea can be desorbed from graphene-based materials; and

FIG. 9 is a plot of nitrogen BET surface area vs. mg of urea bound per gram of sorbent for various activated carbons and an embodiment of graphene oxide.

### DETAILED DESCRIPTION

In one aspect, graphene-based materials are used to sequester urea from aqueous fluids such as blood plasma. It is believed that the urea is sequestered from the fluid via intercalation with the graphene-based material host. As used herein “graphene-based materials (GM)” are two-dimensional (2-D) carbon materials including but not limited to graphene, single layer graphene, multilayer graphene, graphene aggregates, graphene oxide, graphite oxide, reduced graphene oxide, reduced graphite oxide, and exfoliated graphite. GM also includes any and all three-dimensional (3-D) materials made all or in part from 2-D materials. It also means any and all sp² hybridized carbon materials described in “All in the graphene family—A recommended nomenclature for two-dimensional carbon materials” Carbon 65 (2013), 1-6.

Fluids containing urea (CH₄N₂O) can be contacted with the graphene-based material in a number of ways including, for example, dispersing or suspending the graphene-based material in the fluid, passing the fluid through a bed comprising graphene-based material or passing the fluid through a tube coated with graphene-based material. As used herein, an aqueous fluid is a fluid in which the primary liquid carrier is water. For example, 25° C. and atmospheric pressure, the liquid portion of an aqueous fluid, after removal of total dissolved and undissolved solids, is greater than 50% water by weight and, in some embodiments, is greater than 75%, greater than 90%, greater than 95%, greater than 99% or greater than 99.9% water by weight. The graphene-based materials may be in loose particulate form, in a monolith, or may be fixed to a substrate. The graphene-based materials may also be associated with other particles or compositions such as carbon black, activated carbon or indicator compounds. Using GM sorbents, concentrations of urea (mg/L) in biological fluids, such as blood, can be reduced by, for example, greater than 50%, greater than 75%, greater than 90% or greater than 95%. The same fluids may have urea levels reduced to less than 0.5, less than 0.1 or less than 0.01 g urea per liter of fluid. In non-biological fluids, e.g. purified water, GM sorbents can reduce urea concentrations, for example, to parts-per-million levels, or parts-per-billion levels, or parts-per-trillion levels. In many instances, graphene-based materials can sorb urea (mg urea per g of graphene-based material) at greater than 100 mg/g, greater than 200 mg/g, greater than 500 mg/g or greater than 700 mg urea/g of graphene-based material. The graphene-based material may be used in combination with other materials that may be useful in removing additional constituents from a fluid. For example, graphene-based materials can be used in combination with activated carbon to remove a large variety of undesirable materials from blood. In some cases, treatment with GM and activated carbon (or other purifying materials) may be in series where the fluid is treated first by one of the materials and then by the other. In other cases, the graphene-based materials and activated carbon may be mixed or comingled so that different treatments occur concurrently at a single location. After sorbing a quantity of urea, some GM/urea complexes can be recharged by removing some or all of the urea from the complex. In some embodiments, these recharged materials can be re-used. Specific GM/urea complexes can include graphene/urea, oxidized graphene/urea and oxidized graphite/urea. These complexes may include other compounds that have been sorbed from a fluid, but in some cases, urea is the primary, if not the only, compound that is sorbed from a fluid containing urea and other materials. In some cases, a GM/urea complex may contain less than 10%, less than 5%, less than 1% or less than 0.1%, by weight, of compounds other than GM and urea. In other embodiments, additional materials may be sorbed and may account for more than 0.1%, more than 1%, more than 5% or more than 10% of the mass of the GM/urea complex.

Removal of urea from blood is one of the primary roles of the kidney. In the case of end stage renal failure, kidney function is abrogated or completely eliminated, and external means are required to lower urea concentration in blood. In such patients with chronic kidney disease and/or end stage renal disease and/or temporarily or permanently non-functional kidneys and in need of treatment, urea concentrations can be quite high, reaching millimolar concentrations, and in the course of a day, up to 25 grams of urea must be removed from circulation by one or another means. In other cases, approximately 1 gram of urea, or greater than or equal to 2, 3, 4, 5, 6, 7, 8, 9, 10, 11, 12, 13, 14, 15, 16, 17, 18, 19, 20, 21, 22, 23, or 24 grams of urea are removed per day per patient. The primary method for urea removal in patients with kidney failure is dialysis, via hemodialysis or peritoneal dialysis. A portable or wearable artificial kidney that can efficiently remove urea from blood would be a significant advance over current methods.

In industries requiring ultrapure water (e.g. semiconductor wafer fabrication and pharmaceuticals), urea concentrations of even a few parts per billion are considered high. Accordingly, methods have been developed to remove even trace quantities of urea present from water that has already been highly purified. Many of these methods are costly to install and maintain, and the materials and methods disclosed herein can provide more efficient and effective removal of the trace quantities of urea that typically remain in purified water

Although urea is primarily a waste product, it does have value as a source of nitrogen. Nitrogen fertilizers, for example, are in use worldwide. The high nitrogen content of urea in urine (on a mass basis, 46%) makes it attractive as a potential source of fertilizer, and in some cases, the graphene-based materials disclosed herein can provide an economical technique for isolating urea from animal urine. Removal of urea from urine using these graphene-based materials can also provide a source of clean water in applications, such as outer space, where water is scarce.

While urea itself is odorless, it reacts with enzymes in urine to form odiferous compounds. Sequestration of urea, particularly from animal urine, can reduce the odor from pet or livestock urine by rendering it unavailable to the enzyme active sites.

In addition to sequestering urea, some embodiments of the GM described herein can be used to provide a controlled release of urea in certain environments. In agriculture, for example, where urea is a critical source of nitrogen, there is a need to release material over extended periods.

The materials described herein may also be useful in recovering metal ions such as cesium. In some cases, the inclusion of urea can improve the amount of metal ion that is intercalated in the GM.

In some embodiments, the intercalation of urea in graphene-based materials can help lead to exfoliation of the materials without the harsh chemical conditions that are typically required for exfoliation. For example, urea may be intercalated into layered graphenes, graphene oxides, reduced graphite oxide, graphene aggregates, or partially exfoliated graphite to help exfoliate and process these materials.

Intercalation of urea in graphene-based materials may also aid in modifying material properties such as rheology or conductivity. By delaminating or exfoliating, these materials may exhibit increased viscosity or decreased conductivity. Alternatively, urea intercalation in graphene-based materials can help to tune the properties of the materials and may lead, for example, to increased or decreased conductivity or increased or decreased viscosity.

The GM described herein, including graphene, oxidized graphene and oxidized graphite, may be comprised of carbon sheets that are one atom thick. As a result, these materials have very high aspect ratios and the length to thickness aspect ratio of the GM can be greater than 100, greater than 1,000 or greater than 10,000.

### Techniques for Urea Removal

Most known methods for removing urea from solution involve chemical alteration or destruction of urea rather than sequestration of the urea molecule itself. For example, the enzyme urease catalytically decomposes urea to ammonia (ammonium ion) as follows:

(NH₂)₂CO+H₂O→CO₂+2NH₃

Likewise, transition metal catalysts, such as those based on Ni²⁺ coordination complexes, are also able to react with urea.

Urea can be electrochemically oxidized; under some conditions, the products are identical to those generated by the action of urease, i.e. carbon dioxide and ammonia. Urea can be removed from water using so-called advanced oxidation methods that typically comprise chemical or UV or combined treatments.

Known methods of urea destruction such as catalytic decomposition to ammonia, incomplete electrochemical oxidation, and advanced oxidation methods typically produce non-gaseous products such as ammonium ion in water that must also be removed.

Graphite is an allotrope of carbon that consists of layers of sp² hybridized carbon atoms that are stacked and held together by Van der Waals forces. Because of its anisotropy, this form of carbon has found many uses. The single layer of hexagonally packed carbon atoms that form graphite is known as graphene. Materials based on few layered graphites (FLG) or graphene-based materials offer a unique combination of properties. Graphene and graphite may be oxidized to produce materials such as graphite oxide and graphene oxide (the single layer that when stacked forms graphite oxide). Graphite oxide and graphene oxide include oxygen atoms and typically have an atomic ratio of carbon to oxygen of greater than 1.5. In some embodiments, a graphene oxide or graphite oxide sorbent has a carbon content (mole %) of at least about 55%, or 60%, or 65%, or 70%, or 75%, or 80%, or 85%, or 90%, or 95%, or 99%, or 99.99%. In some cases, the balance of the sorbent is oxygen and the sorbent is void of detectable levels of elements other than carbon, hydrogen and oxygen. In other situations, the balance of the sorbent includes one or more elements selected from the group consisting of oxygen, boron, nitrogen, sulfur, phosphorous, fluorine, chlorine, bromine and iodine. In some embodiments, a graphene oxide or graphite oxide has an oxygen content, on a molar basis, of at least about 0.01%, or 1%, or 5%, or 15%, or 20%, or 25%, or 30%, or 35%, or 40%, or 45%. For example, a graphene or graphite oxide sorbent can have a carbon content of at least about 55% and an oxygen content of at least about 0.01%. The oxygen content can be measured with the aid of various surface, titrimetric, or bulk analytical spectroscopic techniques. As one example, the oxygen content is measured by x-ray photoelectron spectroscopy (XPS).

In some embodiments, a GM sorbent comprises or consists of an oxide of graphene-based material (GM oxide) having a bulk carbon-to-oxygen molar ratio of at least about 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1, or 1,000,000:1. In some cases, the GMO sorbent has a surface carbon-to-oxygen ratio of at least about 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1.

In some embodiments, a GM sorbent comprises or consists of graphene oxide (i.e., oxidized graphenes obtained, for example, by exfoliating graphite oxide or by oxidizing graphenes), reduced graphene oxides (i.e., the product of reducing graphene oxides or graphite oxides), or graphite oxide with a bulk carbon-to-oxygen molar ratio of at least about 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1. In some cases, a graphene oxide or graphite oxide-containing sorbent includes graphene oxide or graphite oxide with a surface carbon-to-oxygen ratio of at least about 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1. In some embodiments, the carbon-to-oxygen atomic ratio of the graphene oxide ranges from 1.5:1 to 5:1, from 1.5:1 to 2:1, from 1.5:1 to 3:1, from 2:1 to 5:1, or from 3:1 to 5:1. Graphene oxides can be reduced by various methods, e.g., chemically, thermally, etc. In certain embodiments, reduced graphene oxides have a carbon-to-oxygen molar ratio of at least 5:1. In other embodiments, reduced graphene oxides have a carbon-to-oxygen molar ratio ranging from 2:1 to 1000:1, from 2:1 to 100:1, from 2:1 to 20:1, from 2:1 to 10:1, from 3:1 to 1000:1, from 3:1 to 100:1, from 3:1 to 20:1, from 3:1 to 10:1, from 5:1 to 1000:1, from 5:1 to 100:1, from 5:1 to 20:1, or from 5:1 to 10:1. It is believed that the oxygen atoms are bonded to the graphite or graphene by either single covalent bonds to two adjoining carbon atoms or as singly bonded hydroxyl groups. These graphene-based materials may contain other heteroatoms but in many cases are void of elements other than carbon and oxygen and may contain less than 1%, less than 0.1% or less than 0.01% (by weight or molar) of elements other than carbon, hydrogen and oxygen. In some embodiments, the GM oxide includes at least one organic surface moiety, such as an alkyl group, aryl group, alkenyl group, alkynyl group, hydroxyl group, epoxide group, peroxide group, peroxyacid group, aldehyde group, ketone group, ether group, diketone group, triketone group, anhydride group, lactone group, ester group, carboxylic acid or carboxylate group.

Graphite oxide (GO) can be synthesized using several reactions known to those of skill in the art, such as the Brodie, Staudenmeier and Hummers methods. These processes differ in both the types of oxidizers that are used for the oxidation and the processing conditions. The Brodie method uses a combination of fuming nitric acid and potassium chlorate as the oxidizing agent. The Staudenmeier method uses a combination of concentrated nitric and sulfuric acid and potassium chlorate as the oxidizer. The Hummers method uses potassium permanganate and sulfuric acid. All of these methods produce materials that are chemically similar elementally in that the atomic C:O ratios of the graphite oxides are approximately 2:1.

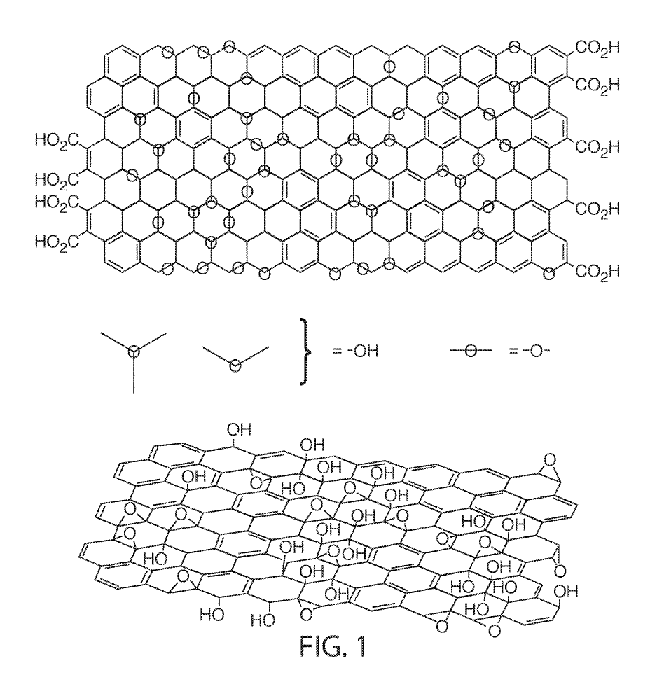

There are many theoretical models in the literature for the structure of graphite oxide, and there is currently no universally agreed upon structure. However, there is more consensus concerning the kinds of functional groups that exist on the surface of GO. These are illustrated in FIG. 1. “From Conception to Realization: An Historical Account of Graphene and Some Perspectives for Its Future” D. R. Dreyer, R. S. Ruoff and C. W. Bielawski, Angewandte Chemie International Edition 49, 9336-9344 (2010). In most species, the oxygen exists on the basal plane in either hydroxyl or epoxy groups. Some models indicate the presence of carboxylic acid groups on the edges of the basal planes.

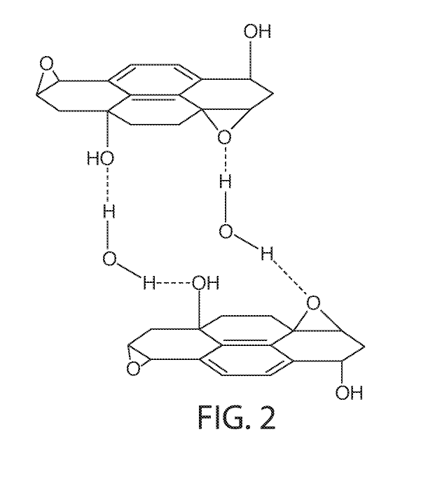

This rich chemical functionality of the GO planes has been used extensively in the literature for the functionalization of graphite oxide (see Chapter 3 in “Functionalization of graphene”, 2014, included herein by reference). The chemistry of GO planes opens up the material to different functionalities but also makes graphite oxide thermally unstable. When GO is heated above 120° C. it decomposes exothermically and releases CO and CO₂ gases which force the basal planes apart and lead to the production of thermally reduced GO. The presence of these functional groups on the basal plane is believed to be responsible for the material's dispersibility in, and strong affinity for, water, as GO can form strong hydrogen bonding networks, as shown in FIG. 2. Id. Graphene Oxide can also be reduced using chemical reduction agents. For instance, graphene oxide can be reduced to graphene powder by using urea, but the reduction process fails to leave any detectable urea or nitrogen associated with the graphene, and there is no formation of a GM/urea complex. See U.S. Patent Application Publication No. 2013/0302693.

“Graphene” as used herein comprises stacked sheets, in which each sheet comprises sp²-hybridized carbon atoms bonded to each other to form a honeycomb lattice. In one embodiment, the graphene comprises few-layer graphenes (FLG), having 2 or more stacked graphene sheets, e.g., a 2-20 layer graphene. In another embodiment, the FLG comprises a 3-15 layer graphene. In one set of embodiments, the graphene can include single-layer graphene and/or graphene having more than 15 or more than 20 layers. In some of these embodiments at least 80%, at least 85%, at least 90%, or at least 95% of the graphene comprises 2-20 layer graphene. In another embodiment, at least 80%, at least 85%, at least 90%, or at least 95% of the graphene comprises 3-15 layer graphene.

The dimensions of graphenes are typically defined by thickness and lateral domain size. Graphene thickness generally depends on the number of layered graphene sheets. The dimension transverse to the thickness is referred to herein as the “lateral” dimension or domain. In many embodiments, the graphene has a mean lateral domain size ranging from 0.5 to 10 nm or, more narrowly, from 1 nm to 5 nm.

Graphenes can exist as discrete particles and/or as aggregates. As used herein the term “graphene aggregates” refers to a plurality of graphene particles (FLG) that are adhered to each other. For graphene aggregates, “mean lateral domain size” refers to the longest indivisible dimension or domain of the aggregate. Thickness of the aggregates is defined as the thickness of the individual graphene particle.

In one embodiment, the surface area of the graphene is a function of the number of sheets stacked upon each other and can be calculated based on the number of layers. In some instances, the graphene lacks pores and exhibits no microporosity. The surface area of a graphene monolayer with no porosity is 2700 m²/g. The surface area of a 2-layer graphene with no porosity can be calculated as 1350 m²/g. In another embodiment, the graphene surface area results from the combination of the number of stacked sheets and amorphous cavities or pores. Other examples of graphene can exhibit a microporosity ranging from greater than 0% to 50%, e.g., from 20% to 45%. In some embodiments, graphene has a nitrogen BET surface area ranging from 40 to 1600 m²/g, from 60 to 1000 m²/g, or from 80 to 800 m²/g. In other embodiments, the graphene, or FLG, has a nitrogen BET surface area of greater than 1000 m²/g, greater than 500 m²/g, greater than 250 m²/g or greater than 100 m²/g. In some embodiments, the total pore volume (pores greater than 1 nm measured by nitrogen desorption or mercury porosimetry) of the GM, such as graphene or GO, is less than 2.5 cm³/g, less than 1.0 cm³/g, less than 0.5 cm³/g, less than 0.1 cm³/g or less than 0.01 cm³/g. It is believed that in activated carbons neither surface chemistry nor pore size distribution factor alone in predicting sorbent performance. Rather, it is a combination of pore size distribution and surface chemistry that dictates the kinetics and thermodynamics for adsorption.

GM may be used freshly prepared or may be aged. For example, under certain conditions, aging can alter the amount of oxygen on carbon surfaces of graphene oxide. Aging can also alter the state of platelet or particle aggregation or agglomeration.

Synthesized graphene or graphite based materials may be of greater purity than carbonaceous compounds, such as activated carbon, that are derived from natural sources. In some applications such as medical and pharmacological processes this level of purity can be critical. For example, in a dialysis application, the sorbent is potentially in contact with materials that are or will be in circulation in the patient's body. Thus, it is important that the leachable content of a sorbent, either organic or inorganic, be kept to an absolute minimum. Likewise, in pharma and semiconductor applications, where ultrapure water is required, release of materials from the sorbent must be kept below parts per billion, or even parts per trillion, levels. Thus, graphene-based materials may be able to meet stringent medical, pharmacological or semiconductor requirements while activated carbons or other naturally derived carbonaceous materials may not.

When dispersed in a urea containing fluid, the GM can be provided in a quantity and concentration that efficiently removes urea from the fluid without adversely affecting fluid dynamics. In some embodiments, the GM can be dispersed or suspended in the fluid at a concentration range of, for example, 0.1 to 100 mg/mL, 0.1 to 10 mg/mL, 1 to 10 mg/mL or 1 to 100 mg/mL. A urea sequestration process may be a continuous or a batch process, and GM/urea complex can be separated from the fluid by methods known to those of skill in the art, such as filtration and centrifugation. In some cases, the fluid comprising urea is passed through a bed comprising GM. The flow rate should provide for efficient removal of urea without resulting in excessive packing of the GM that would result in a significant pressure increase. Flow rates through a bed of GM may be greater than 100 mL/g/min, greater than 1 L/g/min or greater than 10 L/g/min. The same fluid may be passed through a bed once or multiple times, and the fluid can be cycled through the bed multiple times until equilibrium is approached or reached. A GM filter bed may include materials that help prevent the GM from blocking fluid flow. These materials may include other carbonaceous materials such as activated carbon or may include inorganic materials that can be either active or inert. Inorganic materials may include, for example, glass beads or metal oxides such as silica or alumina.

### Urea Binding Mechanisms:

Urea is but one of many species that need to be controlled or removed in patients with chronic or end stage renal disease. In addition to maintaining fluid and electrolyte balance, the kidney is responsible for removal of a variety of toxins. Accordingly, there are a variety of means and/or materials used to replicate, replace or simulate these functions; a sorbent for urea can be associated with one or more of them. Because GM can be made with levels of microporosity that allow the materials to be used for ultrafiltration a GM sorbent can either be associated with the hollow fiber membranes used in ultrafiltration or can itself form a hollow fiber membrane used in ultrafiltration. Thus, in addition to acting as a urea sorbent, the GM can physically (not by intercalation) block molecules larger than urea, such as proteins, while still allowing the passage of water and dissolved ions. This can provide for the sequestration of urea, water and ions from the higher molecular weight components in a fluid that are found, for example, in blood fluids. The sorbent might occupy spaces between fibers in fiber bundles, or could be composites of sorbent and fiber, such that it comprises a single entity. Alternatively, a urea sorbent can be associated with a sorbent targeting a different function (e.g. iron oxy hydroxide that is used for phosphate binding).

Likewise, a urea sorbent could be associated with materials used to control ionic composition, ionic strength, or pH, remove other toxins (e.g. the so-called “middle molecules”. This association could be in the form of physical mixture of the two (or more) sorbents, or it could be a segregated assembly such that the sorbents are stacked on top of one another, as in the REDY device, (as described in http://www.advancedrenaleducation.com/GeneralTopics/HistoryofSorbentTechnology/tabid/587/Default.aspx and

http://www.renalsolutionsinc.com/howitworks.html and references therein, both accessed 2 Feb. 2015). Novel urea sorbents could be associated with zirconium phosphate, zirconium oxide, zirconium carbonate, particles with immobilized urease, Resonium A, sevelamer carbonate, iron oxide hydroxide, zirconium carbonate, or other materials including but not limited to those described in Wester et al., Nephrol. Dial. Transplant (2013) 0: 1-8, the entirety of which is incorporated herein.

An alternative mode of association is a core-shell particle. For example, a particle of iron oxide hydroxide could be coated with a submonolayer, monolayer, or multilayer of particles comprising a novel urea sorbent. The core and shell can be reversed, such that a layer of iron oxide hydroxide could be used to coat a particle of a novel urea sorbent. Core-shell particles are well known in the scientific literature, and there are a variety of methods available to make core-shell particles.

Likewise, there are numerous other geometries by which a novel urea sorbent could be associated with another particle or material used in the care of patients with kidney disease. For example, if one or another of the particles is 2-D, it can be coated with the other material to make a stacked layer. Such a particle could remain in a 2-D geometry (with one material on the top and another on the bottom), or it can be “rolled” or otherwise converted into a 3-D material. Yet another non-limiting example of association is so-called “Janus” particles, where, for example, each material largely occupies one hemisphere of a sphere. Those skilled in the art will recognize a variety of other methods of association.

### Covalent Attachment

One way to improve a sorbent's ability to bind urea is to introduce organic functional groups on the sorbent surface that are capable of forming covalent bonds with urea. This technique can be applied to polymers which can include functional groups like those in ninhydrin or glyoxal. In some cases, covalent single or double bonds can be formed between any of the four elements C, N, H and/or O, such as C—C bonds, C—N bonds, C—O bonds, N—O bonds, N—N bonds, or O—O bonds. Non-limiting examples of functional groups that may bind urea are epoxides, lactones, ketones, hydroxides, alkenes, imines, and alcohols.

Organic functional groups may originally exist on the sorbent (e.g., GM or AC) surface (e.g. C═C or C—C bonds in activated carbon) or can be introduced onto the surface by a separate chemical or physical processing step. Examples of a chemical processing step would be oxidation or reduction. Examples of physical steps could be heating, cooling, milling, grinding or sonicating. In some cases, a physical step might lead to a chemical reaction (e.g. thermally induced oxidation); in other cases, the physical step might expose otherwise hidden functional groups (e.g. exfoliation of layered materials).

Inorganic functional groups, i.e. metal ions, may also be used to bind urea such that the attachment is through coordination or dative bonds. Non-limiting examples of metal ions that could coordinate to urea, either via lone pairs on oxygen or nitrogen or to the double bond from carbon to oxygen (C═O) are Cu²⁺, Zn²⁺, Mn²⁺, Fe^{2+,3+}, and Co²⁺. Metal ions may be native to the sorbent (as is the case for certain activated carbons depending on the raw material) or can be introduced in a separate process.

GM can be used to make membranes, such as filtration membranes. Other more or less planar, closely related forms such as sheets, papers, felts, and cloths have also been described. In these approaches, certain molecules are excluded on the basis of size, while others pass through pores. These materials function in a way similar to conventional filter membranes and are to be distinguished from the sorbents described herein, where molecules are physically and/or chemically bound to the surface of a porous material and are not excluded based exclusively on size.

Adsorption and/or Physisorption

Urea can also bind to sorbents by adsorption and/or physisorption mechanisms. Activated carbon is well known to support both mechanisms. The sites of these interactions may be pores or voids. In the case of activated carbon or carbon black, such pores are usually referred to as macropores, mesopores, and micropores. Pore size can be selected to physically trap specific target molecules, such as urea, between closely-spaced walls of the sorbent material. Atomically scaled layered materials could also present a favorable binding site for urea. For example, a partially exfoliated layer material would generate an accordion-like structure, where a urea molecule might be physically lodged in between the opened-up layers. Such exfoliation might occur naturally, or be generated by a chemical or physical processing step, or by a combination of processes.

With other materials urea immobilization can be obtained by virtue of high surface area. Sorbents with high surface areas will have higher numbers of favorable binding sites. In the case of activated carbon, these sites could be pores, defects in surface structure, or some other site.

A GM can be used in a variety of forms including a powder, a dispersion, a packed bed, a coating or a monolith. In different embodiments the mean lateral domain size of a GM can vary in size and also in size distribution. For example, GM mean lateral domain sizes can vary from 0.005 microns to 10 mm. In particular embodiments, particle sizes may cover the ranges of 0.005 to 0.100 μm, 0.005 to 0.250 μm, 0.005 to 0.500 μm, 0.050 to 0.100 μm, 0.050 to 0.500 μm, 0.050 to 1.0 μm, 0.050 to 10 μm, 0.050 to 100 μm, 0.050 μm to 1.0 mm, 0.500 to 1.00 μm, 0.500 to 10 μm, 0.500 to 100 μm, 0.500 μm to 1 mm, 1.0 μm to 100 μm, 1.0 μm to 1.0 mm, 10 to 100 μm, 10 μm to 1.0 mm, 100 μm to 1.0 mm and 100 μm to 10 mm. At the smallest sizes, the materials could be referred to as colloidal, and could either be solids or dispersed in solution. At the larger sizes, such powders are typically referred to as granular, and at the largest sizes, pellets or extrudates. GM sorbents for urea can thus be colloids, powders, grains, pellets, or extrudates. The distribution in particle could be monodisperse, or bidisperse, or polydisperse. The particles could spherical in shape, or cylindrical, or cubic, or some other regular shape, or could be irregularly shaped. The particles could be two-dimensional in shape (e.g. flakes or sheets). The particles could be isotropic (e.g. spheres), or anisotropic (e.g. cylinders); the aspect ratio of anisotropic particles could be 2:1, or 5:1, or 20:1, or 50:1, or 100:1, or 500:1 (e.g. long needles). In all cases, the particles could be suspended in some other fluid (e.g. water), or be of a gelatinous or foam-like nature, or be used directly as a solid (either dry or wetted). The solid could be free-flowing, or could have restricted flow (i.e. wet, high-aspect ratio flakes).

### Intercalation

Using the materials described herein, urea can be bound to a sorbent through intercalation, the trapping of one species between two or more opposed layers of the sorbing material. Multiple layered materials offer the possibility of binding in between layers, which can significantly increase the effective surface area available for binding. As used herein, a target compound such as urea is “bound” or “sorbed” to a material when the urea preferentially associates with the material in a fluid system in which the target compound is dissolved or dispersed. In different embodiments the urea may be sorbed reversibly or irreversibly. Intercalation hosts that can serve as a sorbent for urea include the graphene-based materials described herein as well as other materials that exhibit similar spacing between opposed layers. For example, materials useful for sorbing urea as intercalation hosts can include any material having two or more opposed layers having interlayer spacing that is properly sized and/or functionalized to capture urea molecules. These materials may be organic or inorganic. In some embodiments, the interlayer spacing between the opposed layers in a host (e.g., GM) useful for intercalating urea is from 2 to 15 Å, 4 to 12 Å, 7 to 11 Å, 8 to 11 Å, 8 to 10 Å, 6 to 9 Å, 5 to 8 Å, 4 to 8 Å, 2 to 8 Å, 2 to 6 Å, 3 to 6 Å, 8 to 12 Å, 9 to 12 Å and 10 to 14 Å.

A wide variety of two-dimensional or layered materials are known in the scientific literature. For example, Miro et al. describe in “At atlas of two-dimensional materials” Chem. Soc. Rev. 2014, 43, 6537-6554, which is incorporated herein by reference herein, a variety of materials including graphene, graphane, fluorographene, chlorographene, silicene, silicane, fluorosilicene, germanene, germanane, fluorogermanene, chlorogermanene, silicon carbide, boron nitride, a-ZnO, a-ZnS, a-ZnSe, a-ZnTe, a-CdO, a-CdS, a-CdSe, a-CdTe, b-ZnS, b-ZnSe, b-ZnTe, b-CdO, b-CdS, b-CdSe and b-CdTe, GaS, GaSe, InS, InSe, HfS₂, HfSe₂, Hffe₂, MoS₂, MoSe₂, MoTe₂, NbS₂, NbSe₂, NbTe₂, NiS₂, NiSe₂, NiTe₂, PdS₂, PdSe₂, PdTe₂, PtS₂, PtSe₂, PtTe₂, ReS₂, ReSe₂, ReTe₂, TaS₂, TaSe₂, TaTe₂, TiS₂, TiSe₂, TiTe₂, WS₂, WSe₂, WTe₂, ZrS₂, ZrSe₂, ZrTe₂, CoCl₂, CoBr₂, FeCl₂, FeBr₂, FeI₂, HfCl₂, HfBr₂, HfI₂, MnCl₂, MnBr₂, MnI₂, MoCl₂, MoBr₂, MoI₂, NbCl₂, NbBr₂, NbI₂, NiCl₂, NiBr₂, TaCl₂, TaBr₂, TaI₂, TiCl₂, TiBr₂, TiI₂, VCl₂, VBr₂, VI₂, WCl₂, WBr₂, WI₂, ZrCl₂, ZrBr₂, ZrI₂, AsCl₃, CrCl₃, CrBr₃, CrI₃, FeCl₃, FeBr₃, MoCl₃, MoBr₃, SbCl₃, ScCl₃, ScBr₃, TiCl₃, TiBr₃, VCl₃, VBr₃, YCl₃ and ZrCl₃. Given what the inventors have found regarding GM as a sorbent for urea, it is believed that one or more of these materials could serve as a sorbent for urea by intercalation. A similar but not identical list of materials is described in Butler et al., “Progress, Challenges, and Opportunities in Two-Dimensional Materials Beyond Graphene” ACS Nano 2013, 7, 2898-2926, incorporated herein by reference.

The inventors also believe that carbides could serve as hosts for urea intercalation. For example, Mashtalir et al. (“Intercalation and delamination of layered carbides and carbonitrides” Nature Communications 2013, 4:1716) demonstrate intercalation of urea into Ti₃C₂(OH)ₓO_{y}F_{z}. This material is one of a large class of two-dimensional materials, and it is believed that many others (see, for example, Naguib, M. et al. “Two-dimensional transition metal carbides” ACS Nano. 6, 1322-1331 (2012) will also demonstrate similar behavior.

Clays, being layered materials, could also be used as intercalating sorbents for binding and release or delivery of urea. For example, Muiambo et al. (Applied Clay Science 2015, 105-106, 14-20) have prepared urea-expanded vermiculite. Yan et al. (American Ceramic Society Bulletin 2005, pp. 9301-9305) describe kaolinite-urea intercalation composites. Kim et al. [J. Soils Sediments (2011) 11:416-422] report on urea intercalation into montmorillonite.

### Clathrates

Urea can form clathrates with other molecules. Clathrates are also referred to as molecular inclusion compounds. Urea molecules can self-assemble around long chain fatty-acid type molecules, or other linear polar hydrocarbons, in a helical structure held together by extensive hydrogen bonding. These clathrates may be stable in aqueous systems and in some cases are reversible. Clathrates can provide for efficient sequestering of urea for several reasons. First, the presence of hydrogen bonding means that the urea molecules are in close proximity, essentially as close-packed as possible. This can lead to the maximum coverage per unit surface area. Sorbents that are able to bind hydrogen-bonded n-mers of urea (where n=2 to 100) will necessarily have a higher capacity than do conventional sorbents.

There are a number of different types of hydrogen bonding, and urea can participate in 3-center hydrogen bonds and/or bifurcated hydrogen bonds. Likewise, as described in J. Phys. Chem. B 2007, 111, 6220-6228 and Spectrochimica Acta Part A 61 (2005) 1-17, both of which are by reference incorporated fully herein, urea can exist in hydrogen-bonded aggregates, and such aggregates might be present within GM sorbents that contain urea, either in pores with sizes that match aggregates, or in between layers via intercalation, or in clathrate-type structures, or in some other type of structure. Such structures might be formed after binding of a single urea molecule (i.e. a self-assembled structure), or could obtain by binding of pre-formed hydrogen-bonded urea aggregates. The affinity of hydrogen-bonded urea aggregates might be substantially greater for particular sites on the GM than for the corresponding urea monomers. This may increase the molar concentration of urea that is sorbed to, or otherwise associated with, a GM, and may allow for a greater concentration of urea than would be theoretically possible based on a monolayer of urea covering the GM.

A sorbent functionalized with polar hydrocarbons in a structure or location or environment that enables assembly of ureas around individual hydrocarbon molecules would potentially have a very high sorption capacity. It is important to note that hydrogen bonded n-mers of urea can be but need not be in a helical geometry.

### Forms of GM

GM can be incorporated as part of devices targeted at any of the applications described herein and known to those of skill in the art. GM can be contained in a bag, flask, tank or other fluid container, as packing in a column, as a coating on a column, either comingled with or separate from other materials. For example, GM can be mixed with activated carbon (AC) for removal of all middle molecules and urea. Alternative, GM can be positioned upstream or downstream of AC in a column targeting toxin removal. GM can be positioned in a device with other sorbents, including metal oxides such as alumina and silica, clays, silicates, metal organic frameworks (MOFs), activated carbon, activated charcoal, carbon black, zeolites, polymers and other known sorbents.

In any of the above, GM can be coated, functionalized, adsorbed to, or otherwise modified with a biocompatible polymer or material, so as to reduce or eliminate any adverse consequence when in contact with biological fluids or organisms, either in vivo or when applied in extracorporeal devices and procedures.

All or part of the device that incorporates GM can be reusable, regenerable, or disposable. If disposable, it can be part of a disposable cartridge or device that can be used for more than 1, 2, 4, 6, 8, 12, 16, 24, 36, 48, 72, 96, 120, 144, or 168 hours.

### Applications

In many embodiments, graphene-based materials can be used to sorb urea outside of the body (extracorporeal). These extracorporeal treatments can include, for example, hemoperfusion, hemodialysis, peritoneal dialysis, hemofiltration, plasmapheresis, ultrafiltration, hemodiafiltration and/or combinations of these methods. The physical principles governing the movement of species in the above processes can be diffusion, convection, electrophoresis, dialectrophoresis, laminar flow, turbulent flow, or any combination thereof. The treatments can involve portable, semi-portable, disposable and/or wearable systems. The biological fluids that the GM can sorb urea from include blood fluids as well as other biological fluids. Blood fluids include those fluids comprising or obtained from blood, for example, whole blood, blood plasma, processed blood, preserved blood, serum, plasma, clotted blood, anti-clotted blood, centrifuged blood and hematocrit. Other biological fluids that may benefit from GM sorbents include filtrate, ultrafiltrate, dialysate, extracellular fluids, intracellular fluids, interstitial fluids, lymphatic fluids, transcellular fluids, urine, urine-derived fluids, or other biologically-derived fluids, including but not limited to kidney or liver dialysate. The use of graphene-based materials can improve the function of known devices that incorporate carbon based sorbents. These devices and systems include, for example, BioLogic-DT®, Hemocleanse, MARS®, Prometheius®, the REDY system (Renal Solutions), the Fresenius PAK (portable artificial kidney) and the Sorbent Management for Advanced Renal Replacement Therapy system. GM can also be used with portable and/or wearable artificial kidneys or related products such as Dialisorb (Renal Solutions Inc.), and those developed by AWAK, Blood Purification Technologies Inc., and other companies. Likewise, GM can be used in conjunction with, or as part of, any of the additional products, devices and designs mentioned in “Wearable Devices for Blood Purification: Principles, Miniaturization, and Technical Challenges”, by Armignacco et al. (Seminars in Dialysis-2015, Wiley Periodicals Inc., pp. 1-6), which is incorporated fully herein by reference.

Another application for urea sorbents is to capture urea from the vapor phase. Urea is a large-volume industrial chemical, and is manufactured globally. It is sold in both solid and liquid forms. In the solid form, it is sold typically in prills or granules, while in liquid form, it is provided as an aqueous solution. In all cases, there is a finite vapor pressure, e.g. 1.2×10⁻⁵ mm mercury (Hg) at 25° C.

Some applications of urea (either a solid or in aqueous solution) include as: a component of fertilizer; a component of animal feed; a reductant in selective catalytic reduction (SCR) systems to lower emissions of nitrogen oxides from stationary and mobile sources (e.g. automobiles); a viscosity modifier for starch or casein-based paper coatings; a component in consumer goods; a stabilizer in explosives; a food additive; an insect repellent; a flavoring agent; a humectant and dehydrating agent; a component of adhesives; a component of polymers; and a component of flame-proofing agent.

A high-performance urea sorbent would be invaluable to prevent workplace exposure, including, for example, oral exposure, inhalation exposure, and/or dermal exposure during the manufacture, packaging, distribution, or use of urea in solid or liquid (aqueous) form.

In selective catalytic reduction (SCR) systems, urea is introduced as a reducing agent into combustion effluent at high temperature to react with nitrogen oxides (NOx). The use of urea as a reductant for NOx reduction in engines is widespread. It would be advantageous to be able to store (and release as needed) the maximum amount of urea in the minimum volume, or the minimum mass, or both. A high performance solid urea sorbent for SCR could serve as a replacement for the current liquid (aqueous) storage, where the urea concentration is roughly 32%. With a solid sorbent, any required water vapor could be drawn directly from the atmosphere or from other sources.

### EXAMPLES

### Example 1

Reagents:

Urea (Sigma Aldrich, ACS Reagent grade), absolute ethanol (Sigma Aldrich, Pure 200 proof), sulfuric acid (Sigma Aldrich, 99.999% purity), 4-(dimethylamino)benzaldehyde (Sigma Aldrich, 99%), 17 MOhm deionized water.

### Urea Calibration Curve—

A 20 mM Urea stock solution was made using 17 MOhm deionized water. A series of urea calibration standards with concentrations 1 mM, 2 mM, 3 mM, 4 mM and 5 mM were made in 17 MOhm. A PAB reagent solution containing 4% (w:v) of 4-(dimethylamino)benzaldehyde and 4% (v:v) sulphuric acid in absolute ethanol was made according to the literature for the assay. The PAB reagent was stored in a dark space when not in use. A calibration curve of absorbance vs urea concentrations was generated using previously prepared urea calibration standards. The sample for evaluation of urea capturing capacity was prepared by pipetting 25 mL of the 20 mM Urea solution into a glass vial containing 1 g of sample. The vial was shaken overnight on a rotary shaker. The dispersion was filtered using a syringe and Millipore PVDF syringe filter, size 0.45 um. An aliquot of sample filtrate (0.5 mL), PAB reagent (0.5 mL) and 17 MOhm water (1.5 mL) were dispensed into a disposable plastic cuvette and mixed thoroughly. The cuvette was capped and the solution was left to incubate for 20 minutes in a light blocking container prior to measuring against the reference sample on the UV/Vis Spectrophotometer. Samples were prepared in duplicate. The absorbance at 422 nm was measured and recorded. The recorded absorbance was used to determine the concentration of Urea in filtrate based on the established calibration curve.

To measure urea binding, a series of carbon-based materials were introduced to a solution of urea in water and shaken overnight, at ambient temperature. The supernatant was filtered through a Millipore PVDF syringe filter, size 0.45 microns, and the remaining urea in solution was quantified by uv-vis spectrophotometry as per above. Table 1 below shows the data. GCN™ 1240 plus, ROX™ 0.8 and DARCO™ 20x50 are all activated carbons available from Cabot Norit.

TABLE 1

| | Inital | Final | Conc. Urea | mg of | Carbon | mg of |
|---|---|---|---|---|---|---|
| | Urea Conc. | Urea Conc. | Removed | Urea | Weight | Urea/g |
| Sample | (mM) | (mM) | (mM) | removed | (mg) | of Carbon |
| Cabot/Norit GCN 1240 plus | 20 | 15.2 | 4.8 | 7.2 | 1000.3 | 7.2 |
| Cabot/Norit ROX 0.8 | 20 | 15.1 | 4.9 | 7.3 | 1000.6 | 7.3 |
| Cabot/Norit ROX 0.8 | 20 | 15.1 | 4.9 | 7.4 | 1000.6 | 7.4 |
| Darco 20X50 | 20 | 16.4 | 3.6 | 5.4 | 1000.5 | 5.4 |

### Example 2: (Preparation of Graphite Oxide Suspension)

70% Nitric acid (19 mL) was placed inside a 100 mL jacketed cylindrical flask connected to a circulation chiller set at 17° C. A magnetic stir bar was used to agitate the acid. 96% sulfuric acid (37 mL) was added in small portions to keep the temperature of the mixture below 30° C. 325 mesh graphite (2 g, from Alfa Aesar) was added to the acid mixture. The mixture was stirred for at least 10 min. to fully incorporate the graphite. The head space over the reaction mixture was purged with nitrogen at a flow rate of 0.5 L/min. 24 g of a 42 wt. % aqueous solution of sodium chlorate was placed inside a 60 mL syringe and injected into the reaction flask at 0.32 mL/min. Upon completion of the addition of the sodium chlorate solution, the chiller temperature was raised to 20° C. Agitation of the reaction mixture was continued for another 12 hours. The resulting suspension was added into a glass beaker containing 600 mL of cold water at 5° C. stirred with an overhead mixer. The graphite oxide crude product was then isolated by vacuum filtration through a Whatman grade 54 filter paper. The collected filter cake was washed with 300 mL of deionized water. The washed material was left in the filter funnel to dry for 30 minutes under vacuum.

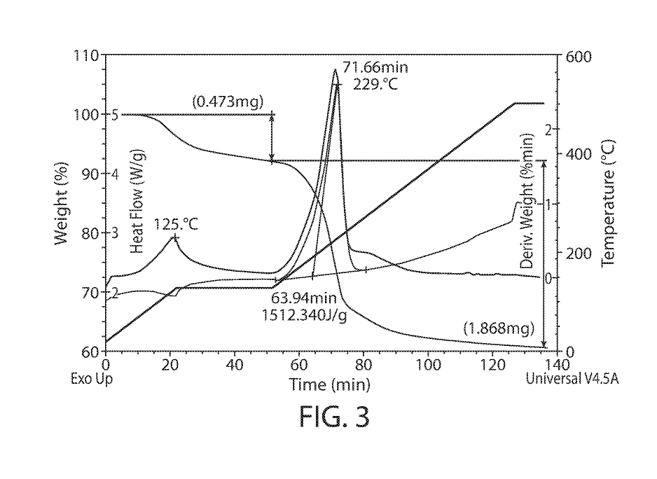

The graphite oxide (washed and dried) was analyzed by thermogravimetric analysis (TGA) combined with differential scanning calorimetry (DSC). The combined scan is shown in FIG. 3 indicating that the graphite oxide contains >30 wt % volatiles, indicating that it is heavily oxidized.

### Example 3: (Preparation of pH 3 Graphene Oxide Suspension)

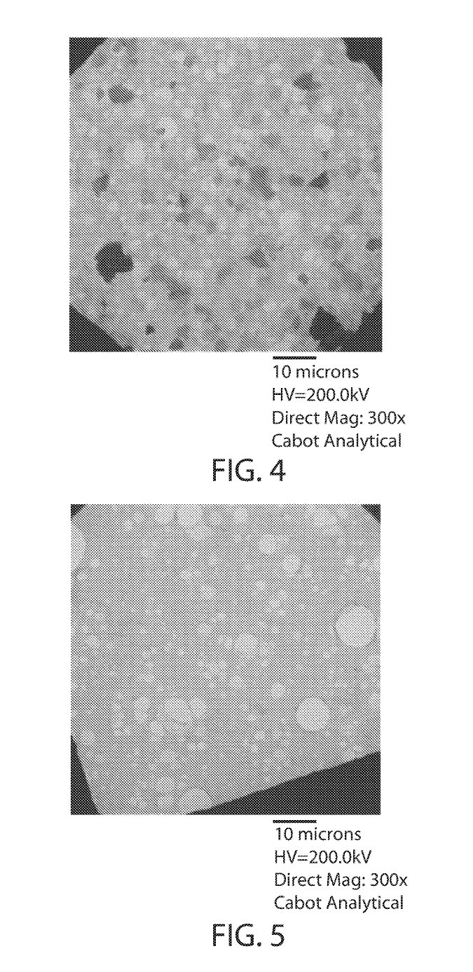

The filter cake of Example 2 was scraped off the filter paper and mixed with deionized water to prepare 125 g of suspension. The suspension was then tip sonicated to exfoliate the graphite oxide into graphene oxide. A TEM image of the pH 3 exfoliated GO suspension is shown in FIG. 4. There is a distribution of thicknesses in the exfoliated GO platelets and the mean lateral size of the platelets is around 10 microns.

### Example 4: (Preparation of pH 9 Graphene Oxide Suspension)

The filter cake of Example 2 was scraped off the filter paper and mixed with deionized water to prepare 640 g of suspension. 1M sodium hydroxide solution was added to raise the pH to 9. The suspension was then tip sonicated to exfoliate the graphite oxide into graphene oxide. A TEM image of the pH 9 graphene oxide suspension (FIG. 5) shows that the platelets are mostly exfoliated and have lateral sizes below 10 microns.

### Example 5

Tables 2 and 3, below, provides data regarding the amount of urea removed from an aqueous sample using known activated carbons as well as graphene-based materials disclosed herein. Note that much larger quantities of the control materials (activated carbon) than GM were required in order to document recordable amounts of urea removal.

TABLE 2

| | | Inital | Final | Conc. Urea | mg of | Carbon | mg of |
|---|---|---|---|---|---|---|---|
| | | Urea Conc. | Urea Conc. | Removed | Urea | Weight | Urea/g |
| Source | Sample | (mM) | (mM) | (mM) | removed | (mg) | of Carbon |
| Cabot | PK 0.25-1 | 20 | 15.7 | 4.3 | 6.4 | 1000.6 | 6.4 |
| Cabot | Norit C GRAN | 20 | 16.5 | 3.5 | 5.2 | 1000.4 | 5.2 |
| Cabot | RX 1.5 Extra | 20 | 14.0 | 6.0 | 8.9 | 1000.0 | 8.9 |
| Electrostal, Russia | FAS-0 | 20 | 14.9 | 5.1 | 7.6 | 1002.6 | 7.6 |
| Kuraray | Kuraray YP17 | 20 | 13.8 | 6.2 | 9.4 | 1000.7 | 9.4 |
| Example 3 | GO pH 2.5 2.5% | 19 | 17.0 | 2.2 | 3.3 | 25.1 | 130.7 |
| Example 4 | GO pH 8.9 0.5% | 19 | 17.1 | 2.1 | 3.2 | 5.0 | 627.9 |

TABLE 3

| | Inital | Final | Conc. Urea | mg of | Carbon | mg of |
|---|---|---|---|---|---|---|
| | Urea Conc. | Urea Conc. | Removed | Urea | Weight | Urea/g |
| Sample | (mM) | (mM) | (mM) | removed | (mg) | of Carbon |
| 1 g ROX 0.8, 24 mL Urea, 2 mL water | 18 | 14.2 | 4.3 | 6.5 | 1000.7 | 6.5 |
| 2 g GO pH 2.5, 2.5%, 24 mL Urea | 18 | 17.8 | 0.6 | 0.9 | 50.2 | 18.9 |
| 1 g GO pH 8.9, 0.5%, 24 mL Urea, 1 mL Water | 18 | 17.7 | 0.7 | 1.1 | 5.0 | 217.7 |
| 2 g GO pH 8.9, 0.5%, 24 mL Urea | 18 | 17.5 | 1.0 | 1.5 | 10.0 | 147.6 |
| 15 mg dried GO, 24 mL Urea, 2 mL water | 18 | 17.6 | 0.8 | 1.3 | 15.2 | 84.0 |
| 1 g ROX 0.8, 26 mL water | 0 | 0.0 | 0.0 | 0.0 | 1000.3 | 0.0 |
| 15 mg dried GO, 26 mL water | 0 | 0.0 | 0.0 | 0.0 | 15.0 | 0.0 |
| 2 g GO pH 8.9, 0.5%, 24 mL water | 0 | 0.0 | 0.0 | 0.0 | 10.0 | 0.0 |

### Example 6

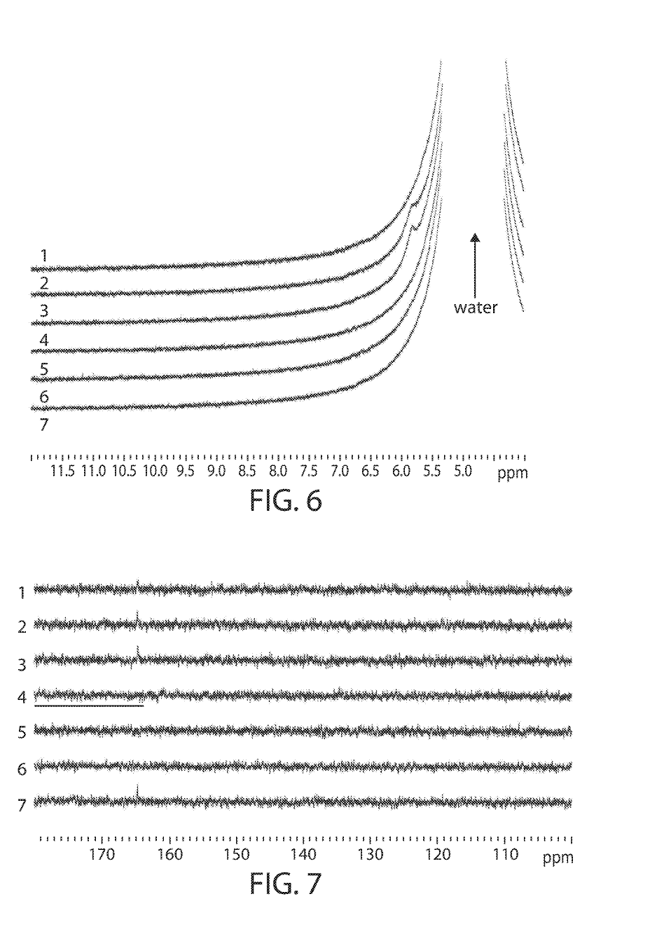

Additional experiments were run with blank samples to see if any artifacts were associated with the removal process. Results show an absence of any artifacts of concern. To show that graphene-based materials actually sequester urea and do not convert it to another species, ¹H and ¹³C experiments were carried out on the supernatants of the materials used in Example 3. Possible decomposition products include hydroxyurea (formed by oxidation), and the condensation products biuret and isocyanic acid. NMR results are provided in FIGS. 6 and 7. NMR Peaks for the ¹H chemical shifts for hydroxyurea (approx. 7 ppm), biuret (approx. 8.3 ppm) and isocyanic acid (approx. 9.1 ppm) were not observed; the only peaks observed were those for urea (approx. 5.9 ppm), and the large water peak. No resonances were observed upfield. Consistent results were obtained from the ¹³C NMR spectrum, where no peaks were obtained other than those for urea. The results indicate the absence of any measurable species in solution other than urea by either technique. The conclusion is that the disappearance of urea from solution results from adsorption to the carbon materials.

### Example 7

### Preparation of Reduced Graphite Oxide

A graphene oxide filtercake as described above was scraped off the filter paper and vacuum dried at 60° C. overnight. The dry GO powder was then ground and passed through a 1000° C. furnace (purged with nitrogen) to thermally reduce the GO and convert it into reduced GO (rGO) platelets. The thermal reduction process produces materials with much a bulk density of ˜2 g/l with a worm-like morphology.

The elemental analysis of the resulting reduced GO by ICP is summarized in Table 4 below.

TABLE 4

| Element | μg/g by ICP |
|---|---|
| Al | 7.20 |
| B | <5 |
| Ba | <5 |
| Ca | 25.70 |
| Co | <5 |
| Cr | <5 |
| Cu | <5 |
| Fe | <5 |
| K | <5 |
| Mg | <5 |
| Mn | <5 |
| Mo | <5 |
| Na | 840.00 |
| Ni | <5 |
| Si | 14.90 |
| Ti | <5 |
| V | <5 |
| Zn | <5 |
| Zr | <5 |

### Example 8

A graphene aggregate was analyzed to determine surface area (SA) by N₂ BET, lateral domain, and thickness properties. The results are listed in Table 5 below. Graphene A was a graphene aggregate obtained from Cabot Corporation.

TABLE 5

| Sample | SA (m²/g) | Thickness (nm) | Lateral Domain (μm) |
|---|---|---|---|
| Graphene A | 349 | 2.5 | 2 |

The elemental composition of the graphene aggregate was analyzed by ICP. Results for Graphene A are shown in Table 6 below.

TABLE 6

| Element | Graphene A |
|---|---|
| Al | <2 |
| Ba | <2 |
| Ca | 2.00 |
| Co | <2 |
| Cr | <2 |
| Cu | <2 |
| Fe | 3.40 |
| K | <2 |
| Ms | <2 |
| Mn | <2 |
| Mo | <2 |
| Na | <2 |
| Ni | <2 |
| Si | <2 |
| Sr | <2 |
| Ti | 4.80 |
| V | 3.00 |
| Zn | <2 |
| Zr | 2.00 |

### Example 9

Additional sequestration tests were run using reduced graphene oxide (RGO), filtered graphene oxide and centrifuged graphene oxide. Tests were also run using filtered and centrifuged activated carbon (ROX™ Cabot Corp) and mixtures of activated carbon and graphene oxide. The sample preparation for centrifugation is the same as described in the filtration method however the in place of filtration the dispersion was transferred to 50 mL centrifuged vial and centrifuged for 45 minutes at 8000 RPM and 1 hour at 10000 RPM at ambient temperature. Next, a portion of the supernatant was filtered using a syringe and Millipore PVDF syringe filter, size 0.45 um. An aliquot of sample filtrate (0.5 mL), PAB reagent (0.5 mL) and 17 MOhm water (1.5 mL) were dispensed into a disposable plastic cuvette and mixed thoroughly. The cuvette was capped and the solution was left to incubate for 20 minutes in a light blocking container prior to measuring against the reference sample on the UV/Vis spectrophotometer. Samples were prepared in duplicate. The absorbance at 422 nm was measured and recorded. The recorded absorbance was used to determine the concentration of urea in filtrate based on the established calibration curve.

TABLE 7

| | Inital | Final | Conc. Urea | mg of | Carbon | mg of |
|---|---|---|---|---|---|---|
| | Urea Conc. | Urea Conc. | Removed | Urea | Weight | Urea/g |
| Sample | (mM) | (mM) | (mM) | removed | (mg) | of Carbon |
| RGO | 20 | 17.4 | 2.6 | 3.9 | 15.5 | 249.9 |
| 5x 0.5% pH 8.9 GO (Example 4) | 16 | 14.2 | 1.9 | 2.9 | 25.0 | 116.3 |
| 0.5% pH 8.9 GO filtered | 19 | 16.7 | 2.6 | 3.8 | 5.0 | 764.9 |
| Graphene Aggregate | 20 | 17.6 | 2.4 | 3.6 | 25.3 | 142.8 |
| ROX 0.8 filtered | 20 | 13.9 | 6.1 | 9.1 | 1000.4 | 9.1 |
| ROX 0.8 centrifuge | 20 | 14.0 | 6.0 | 9.0 | 1000.6 | 9.0 |
| 0.5% pH 8.9 GO centrifuge (Example 3) | 19 | 16.6 | 2.7 | 4.0 | 5.0 | 790.6 |
| 0.5% pH 8.9 GO/ROX 0.8 | 19 | 16.8 | 2.5 | 3.7 | 5.0 | 736.5 |

### Example 10

Additional sequestration experiments were carried out using graphene aggregates. The results are shown in Table 8.

TABLE 8

| | Intital | Final | Conc. Urea | mg of | Carbon | mg of |
|---|---|---|---|---|---|---|
| | Urea Conc. | Urea Conc. | Removed | Urea | Weight | Urea/g |
| Sample | (mM) | (mM) | (mM) | removed | (mg) | of Sample |
| 50 mg ROX 0.8 | 20 | 19.0 | 1.0 | 1.5 | 50.1 | 30.6 |
| 50 mg graphene aggregate | 20 | 18.5 | 1.5 | 2.3 | 50.7 | 45.4 |
| 25 mg graphene aggregate | 20 | 18.5 | 1.5 | 2.3 | 25.8 | 87.7 |

### Example 11

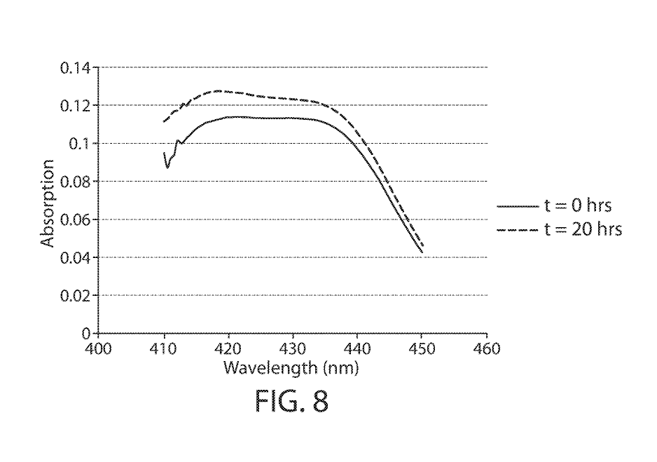

A sample of graphene aggregate (25 mg) that had been exposed to 25 mls. of 20 mM solution of urea overnight was used to demonstrate desorption. Without disrupting the material at the bottom of the sample vial, the urea solution was removed, leaving approximately 1 ml. of solution, and replaced with approximately 3 ml of 17 MOhm DI water, again without disrupting the material. This solution was removed, and replaced with approximately 5 ml. of 17 MOhm DI water. One (1) ml. of the solution was withdrawn and set aside, and the remaining solution was hand shaken and then allowed to sit overnight at ambient temperature. The following day, a sample was withdrawn, and the two samples were analyzed as described above. FIG. 8 shows the UV-Vis spectra obtained for the two samples. Because the graphene sample was not completely separated from the initial urea solution, the initial sample (after addition of the colorimetric reagent) had a non-zero absorbance, indicating the presence of urea. Importantly, the sample tested after 20 hours of exposure shows a greater absorbance, corresponding to an increased concentration of urea in solution. This increase can only be attributable to desorption from the sorbent and demonstrates the utility of these carbon materials for controlled urea release and for the re-use of GM sorbents once, twice, three times, four times or more than four times.

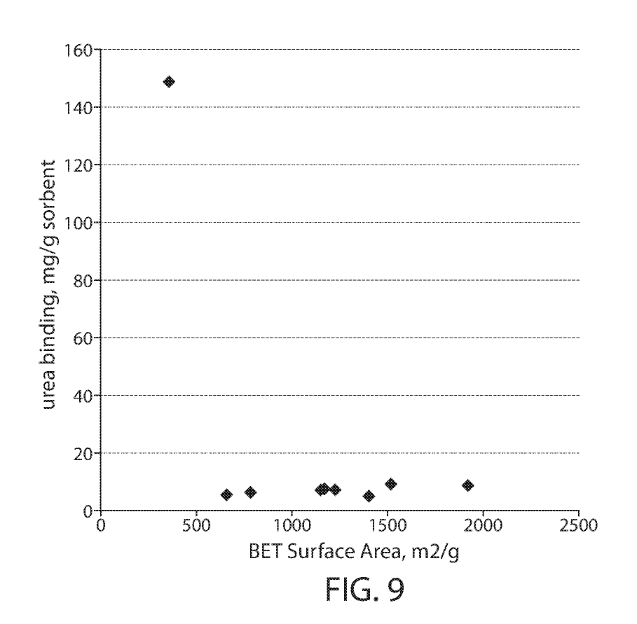

FIG. 9 shows a plot of urea binding (mg/gram sorbent) vs. measured BET surface area (m²/g), taken from the data in Table 9. The data show that surface area has no correlation with binding capacity: the sample with the smallest BET surface area (Cabot graphene aggregates) exhibits a 15-fold improvement in performance vs. the other porous materials. Moreover, the performance does not correlate with particle size. The YP-17D activated carbon, like the Cabot graphene aggregates, is in the micron range of particle size, unlike the other materials in the table, which are in the 0.3-3 mm range. The YP-17D also has significantly higher surface area (4×) than the Cabot graphene aggregates, yet it binds less than 10 mg/urea per g sorbent at ambient temperature. These data clearly show the unique, unanticipated, and special properties associated with layered, 2-D carbon materials for urea binding.

TABLE 9

| | | BET | | |
|---|---|---|---|---|
| | | Surface | Urea Bind | Particle |
| | | Area | (mg per | Size |
| Source | Description | (m2/g) | g sorbent) | (mm) |
| Cabot | GCN 1240 Plus | 1150 | 7.2 | 0.4-1.7 |
| Cabot | ROX 0.8 | 1225 | 7.4 | 0.8 |
| Cabot | DARCO 20x50 | 650 | 5.4 | 0.3-0.8 |
| Cabot | PK 0.25-1 | 775 | 6.4 | 0.5-1.2 |
| Cabot | Norit C GRAN | 1400 | 5.2 | 0.5-1.7 |
| Cabot | RX 1.5 Extra | 1920 | 8.9 | 1.5 |
| Electrostal, | FAS-0 | 1166 | 7.6 | 2.0-3.0 |
| Russia | | | | |
| Kuraray | YP-17D | 1516 | 9.4 | 0.005 |
| Cabot | Cabot graphene | 349 | 148.6 | 0.001-0.0002 |
| | aggregates | | | |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated in their entirety herein by reference.

What is claimed is:

1. A method comprising:
contacting a fluid comprising urea with a mass of graphene-based material particles;
sorbing at least a portion of the urea into or onto the graphene-based material particles to produce a graphene-based material/urea complex; and
reducing the level of urea in the fluid wherein the amount of urea in the graphene-based material/urea complex is greater than 25 mg urea per gram of graphene-based material.

2. The method of claim 1 wherein the fluid is selected from at least one of an aqueous fluid, water, whole blood, blood plasma, processed blood, preserved blood, serum, plasma, clotted blood, anti-clotted blood, centrifuged blood, hematocrit, biological filtrate, ultrafiltrate, dialysate, extracellular fluids, intracellular fluids, interstitial fluids, lymphatic fluids, transcellular fluids, urine, urine-derived fluids, agricultural runoff and sewage.

3. (canceled)

4. The method of claim 1 wherein the concentration of urea in the fluid is reduced by greater than 10 percent by weight.

5. The method of claim 1 comprising agitating, stirring, shaking, sonicating, flowing, cooling and/or heating a suspension of the graphene-based material particles in the fluid.

6. The method of claim 1, the method comprising flowing the fluid through a bed comprising graphene-based material particles.

7. The method of claim 1 wherein the graphene-based material is graphene oxide having an atomic ratio of carbon to oxygen of from 20:1 to 1.5:1.

8. The method of claim 1 wherein the graphene-based material is a graphene aggregate.

9. The method of claim 1 comprising removing at least one non-urea component of the fluid with graphene-based materials, activated carbon and/or modified activated carbon.

10-14. (canceled)

15. A method comprising:
contacting a dialysate with graphene-based material, the dialysate comprising urea;
sorbing at least a portion of the urea on or in the graphene-based material to form a graphene-based material/urea complex;
reducing the concentration of urea in the dialysate by greater than 25%, wherein the graphene-based material/urea complex comprises at least 10% urea by weight.

16. The method of claim 15 further comprising contacting the dialysate with activated carbon.

17. The method of claim 15 wherein contacting the dialysate with graphene-based material is selected from dispersing graphene-based material particles in the dialysate, passing the dialysate through a bed comprising graphene-based material particles, passing the dialysate through a membrane comprising graphene-based material and passing the dialysate through a column comprising graphene-based material.

18. The method of claim 15 wherein the graphene-based material is graphene aggregates or graphene oxide.

19. The method of claim 15 wherein at least a portion of the graphene-based material/urea complex is formed through intercalation.

20. The method of claim 15 further comprising removing urea from the graphene-based material/urea complex.

21. The method of claim 15 wherein the method is used to purify a fluid derived from one or more tissues of a patient exhibiting kidney dysfunction.

22-54. (canceled)
